(19) United States
(12) Patent Application Publication
(10) Pub. No.: US 2024/0230650 A1
(43) Pub. Date: Jul. 11, 2024

(54) METHODS FOR DIAGNOSIS AND TREATMENT OF CHRONIC PROSTATITIS AND/OR CHRONIC PELVIC PAIN SYNDROME

(71) Applicant: NORTHWESTERN UNIVERSITY, Evanston, IL (US)

(72) Inventors: Praveen THUMBIKAT, Evanston, IL (US); Anthony SCHAEFFER, Evanston, IL (US)

(21) Appl. No.: 18/559,244

(22) PCT Filed: May 5, 2022
(86) PCT No.: PCT/US2022/027897

§ 371 Date: Nov. 6, 2023

Related U.S. Application Data
(60) Provisional application No. 63/184,654, filed on May 5, 2021.

(51) Int. Cl. G01N 33/573

(52) U.S. Cl. CPC G01N 33/573; G01N 2333/948; G01N 2333/96433; G01N 2800/342

(57) ABSTRACT

Provided herein and methods and systems for diagnosis of chronic prostatitis (CP) and/or chronic pelvic pain syndrome (CPPS). In particular, methods and systems are provided for monitoring levels of bio-markers of mast cell activity (e.g., mast cell tryptase, carboxypeptidase A3 (CPA3), etc.) in a sample from a subject (e.g., urine, expressed prostatic secretions (EPS), etc.), diagnosing the subject as suffering from CP/CPPS based thereon, and/or treating a subject for CP/CPPS based thereon.

### CROSS-REFERENCE TO RELATED APPLICATIONS

This application claims the benefit of U.S. Provisional Patent Application No. 63/184,654, filed on May 5, 2021, which is incorporated by reference herein.

### FIELD

Provided herein and methods and systems for diagnosis of chronic prostatitis (CP) and/or chronic pelvic pain syndrome (CPPS). In particular, methods and systems are provided for monitoring levels of biomarkers of mast cell activity (e.g., mast cell tryptase, carboxypeptidase A3 (CPA3), etc.) in a sample from a subject (e.g., urine, expressed prostatic secretions (EPS), etc.), diagnosing the subject as suffering from CP/CPPS based thereon, and/or treating a subject for CP/CPPS based thereon.

### BACKGROUND

CP/CPPS is a complex syndrome that is manifested primarily as pelvic pain and is typically a diagnosis of exclusion, rather than being identifiable by known biomarkers. CP/CPPS can be successfully modeled in a murine model of experimental autoimmune prostatitis (EAP). Mast cells are shown to be involved in the pathogenesis of EAP and the pelvic pain response in the murine model is dependent on the presence of mature mast cells.

Tryptase levels in biological fluids have been used as indicators of mast cell number and activation. Mast cell tryptase is a tetrameric neutral serine protease with a molecular weight of 134 kDa. β-Tryptase levels in serum are elevated in most subjects with systemic anaphylaxis. Mast cell tryptase has been demonstrated to be useful in diagnosis of systemic mastocytosis.

### SUMMARY

Provided herein and methods and systems for diagnosis of chronic prostatitis (CP) and/or chronic pelvic pain syndrome (CPPS). In particular, methods and systems are provided for monitoring levels of biomarkers of mast cell activity (e.g., mast cell tryptase, carboxypeptidase A3 (CPA3), etc.) in a sample from a subject (e.g., urine, expressed prostatic secretions (EPS), etc.), diagnosing the subject as suffering from CP/CPPS based thereon, and/or treating a subject for CP/CPPS based thereon.

In some embodiments, provided herein are methods comprising: (a) obtaining an expressed prostatic secretion (EPS) sample from a subject; and (b) having the sample tested to quantify the level of mast cell tryptase in the sample. In some embodiments, methods further comprise diagnosing the subject as suffering from chronic prostatitis (CP) and/or chronic pelvic pain syndrome (CPPS) if the tested level of mast cell tryptase in the sample is elevated above control. In some embodiments, methods further comprise treating the subject for CP/CPPS if the tested level of mast cell tryptase in the sample is elevated above control. In some embodiments, a kit is provided comprising reagents for performing the methods obtaining an EPS sample from a subject and/or testing the sample to quantitate the level of mast cell tryptase in the sample.

In some embodiments, provided herein are methods comprising: (a) obtaining a urine sample from a subject; and (b) having the sample tested to quantify the level of carboxypeptidase A3 (CPA3) in the sample. In some embodiments, methods further comprise diagnosing the subject as suffering from chronic prostatitis (CP) and/or chronic pelvic pain syndrome (CPPS) if the tested level of carboxypeptidase A3 (CPA3) in the sample is elevated above control. In some embodiments, methods further comprise treating the subject for CP/CPPS if the tested level of carboxypeptidase A3 (CPA3) in the sample is elevated above control. In some embodiments, a kit is provided comprising reagents for performing the methods of obtaining a urine sample from a subject and/or testing the sample to quantitate the level of CPA3 in the sample.

In some embodiments, provided herein are methods comprising the steps of: (a) having a urine sample from a subject tested to quantify the level of carboxypeptidase A3 (CPA3) in the sample; and (b) having an EPS sample from a subject tested to quantify the level of mast cell tryptase in the sample. In some embodiments, methods further comprise diagnosing the subject as suffering from chronic prostatitis (CP) and/or chronic pelvic pain syndrome (CPPS) if the tested level of carboxypeptidase A3 (CPA3) and/or the level of mast cell tryptase is elevated above control. In some embodiments, methods further comprise treating the subject for CP/CPPS if the tested level of carboxypeptidase A3 (CPA3) and/or the level of mast cell tryptase is elevated above control. In some embodiments, a kit is provided comprising reagents for performing the methods of testing to quantitate the level of CPA3 and/or mast cell tryptase.

In some embodiments, provided herein are methods comprising: (a) obtaining a biological sample from a subject; (b) having the sample tested to quantify the level of mast cell tryptase in the sample. In some embodiments, the biological sample is an expressed prostatic secretion (EPS) sample. In some embodiments, the subject is suspected of suffering from chronic prostatitis/chronic pelvic pain syndrome (CP/CPPS). In some embodiments, methods further comprise diagnosing the subject as suffering from chronic prostatitis (CP) and/or chronic pelvic pain syndrome (CPPS) if the tested level of mast cell tryptase in the sample is elevated above control. In some embodiments, methods further comprise treating the subject for CP/CPPS if the tested level of mast cell tryptase in the sample is elevated above control. In some embodiments, provided herein are kits comprising reagents for performing the methods herein.

### BRIEF DESCRIPTION OF THE DRAWINGS

FIG. 1. Mast cell numbers increase in EAP. (A) Mast cell in toluidine blue stained sections of mouse prostate harvested at 0, 10, 20, and 30 days after EAP induction. Increased degranulation with time denoted by less intracytoplasmic granular staining of mast cells. The arrows point to mast cells in different stages of activation. (B) Fluorescent stained sections of mouse prostates harvested at 0, 10, 20, and 30 days after EAP induction. The sections were stained with anti-NGF antibody (red) and DAPI (blue). The arrows point to areas of increased NGF expression. (C) Mast cell numbers were determined in non-serial sections of the mouse prostate stained with toludine blue. The total number of mast cells in EAP were categorized as activated, partially activated, and resting. (I) Shows the total amount mast cells during the time course. (II, III, IV) Show the amount of activated, partially activated, and resting mast cells respectively. Scale bar represents 50 μm.

FIG. 2. Chronic pelvic pain is dependent on mast cells. Male B6 (n=10) (A) or KitW-sh/KitW-sh mice (n=8) (B) were tested for tactile allodynia using Von Frey filaments at baseline and 5, 10, 20 and 30 days after EAP induction. (C) Serial sections of the prostate of B6 and KitW-sh/KitW-sh mice following EAP induction were stained using hematoxylin and eosin and scored in a blinded manner for inflammation using standardized criteria. (D) B6 and KitW-sh/KitW-sh prostate sections show foci of inflammation but only B6 mice show the presence of mast cells. Arrows in upper panel indicate inflammatory infiltrates and in lower panels, the presence of mast cells in B6 and its absence in KitW-sh/KitW-sh prostate sections. Scale bar represent 50 μm.

FIG. 3. Tryptase and carboxypeptidase A are increased in CP/CPPS patients. (A) Mast cell tryptase was significantly increased in EPS from CP/CPPS (n=16) patients compared to healthy volunteers (n=6). (B) Urine samples (V3 and V2) collected from CP/CPPS (n=21) patients showed increased presence of carboxypeptidase A (CPA3) compared to control (n=8); (C) however, NGF levels did not change compared to control group.

FIG. 4. Combination mast cell inhibitor treatment in CPPS reduces tryptase levels in EPS and a reduction in the NIH-CPSI and AUA-SI. **p<0.01, ***p<0.0001

### DEFINITIONS

To facilitate an understanding of the present invention, a number of terms and phrases are defined below:

As used herein, the term “subject” refers to any animal (e.g., a mammal), including, but not limited to, humans, non-human primates, rodents, and the like, which is to be the recipient of a particular treatment or subject to various tests (e.g., diagnostic tests) that may be provided by the present invention. Typically, the terms “subject” and “patient” are used interchangeably herein in reference to a human subject.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include, among other things, body fluids (e.g., semen), blood products (e.g., plasma, serum and the like), and their component parts (e.g., expressed prostatic secretions, termed “ESPs” herein, seminal plasma or seminal fluid).

As used herein, the term “subject suspected of having CPPS” refers to a subject that presents one or more symptoms indicative of chronic pelvic pain syndrome (CPPS) or is being screened for CPPS (e.g., during a routine physical). A subject suspected of having CPPS may also have one or more risk factors. A subject suspected of having CPPS has generally not been tested for CPPS. However, a “subject suspected of having CPPS” encompasses an individual who has received an initial diagnosis but for whom the nature of the CPPS is not known. The term further includes people who once had CPPS.

As used herein, the term “subject at risk for CPPS” refers to a subject with one or more risk factors for developing CPPS.

As used herein, the term “characterizing CPPS in a subject” refers to the identification of one or more properties of CPPS in a subject.

As used herein, the term “CPPS marker” or “CPPS marker genes” refers to a biomarker (e.g., gene) whose presence, absence, concentration, and/or expression level (e.g., as detected by mRNA or protein expression or level), alone or in combination with other markers, is correlated with CPPS or prognosis of CPPS. The correlation may relate to either an increased or decreased expression of the gene or amount of the biomarker.

As used herein, the term “subject diagnosed with a CPPS” refers to a subject who has been tested and found to have CPPS. The CPPS may be diagnosed using any suitable method, including but not limited to, the diagnostic methods of the present invention.

As used herein, the term “initial diagnosis” refers to results of initial CPPS diagnosis (e.g. the presence or absence of CPPS).

### DETAILED DESCRIPTION

Provided herein and methods and systems for diagnosis of chronic prostatitis (CP) and/or chronic pelvic pain syndrome (CPPS). In particular, methods and systems are provided for monitoring levels of biomarkers of mast cell activity (e.g., mast cell tryptase, carboxypeptidase A3 (CPA3), etc.) in a sample from a subject (e.g., urine, expressed prostatic secretions (EPS), etc.), diagnosing the subject as suffering from CP/CPPS based thereon, and/or treating a subject for CP/CPPS based thereon.

Experiments conducted during development of embodiments herein demonstrate that mast cell tryptase is useful as biomarker that is elevated in the expressed prostatic secretions (EPS) of patients with CP/CPPS. Similarly, elevated levels of carboxypeptidase A3 (CPA3) were demonstrated to be present in the urine of subjects suffering from CP/CPPS compared to control subjects.

Prostatitis is categorized into five different subsets and accounts for approximately 2 million outpatient visits per year in the United States, including 1% of those to primary care physicians. Of these chronic prostatitis/chronic pelvic pain syndrome (CP/CPPS) is the most common, accounting for 90-95% of all prostatitis diagnoses. This syndrome is most easily distinguished from the remaining categories due to the lack of bacterial association with pain symptoms, as is the case with acute prostatitis (I), or chronic bacterial prostatitis (II). CPPS is further divided by the presence or absence of inflammatory markers in the expressed prostatic secretion (EPS) of affected patients, inflammatory CPPS (IIIa) and non-inflammatory CPPS (IIIb), respectively. Finally, the last subset of prostatic disease is that associated with inflammation of the prostate without emergence of pain symptoms, asymptomatic prostatitis (IV) and is usually diagnosed coincidentally. The impact and burden of CPPS to patients is enormous. Patients suffer considerable morbidity throughout their lives resulting in a significant decrease in quality of life due to the physical and physiological impact. Epidemiological data for CPPS prevalence varies substantially based on method of collection. Estimates suggest as many as 1.6 million U.S. men (or 1.3%) between the ages of 30 and 79 have symptoms of CP/CPPS. Studies involving a medical diagnosis suggest CP/CPPS affects as many as 9% of men while the prevalence of self-reported prostatitis-like symptoms has been estimated to be as high at 16%. Overall patient spending in 2007 for the diagnosis and management of urologic chronic pelvic pain conditions in men (CP/CPPS) and women (interstitial cystitis) exclusive of medication costs for Medicare beneficiaries 65 and older was estimated as $301 million. Factors complicating the management of this condition include its multifactorial pathogenesis, lack of a gold standard for diagnostic testing, and the methodologic limitations of many treatment studies.

Numerous studies have aimed to associate expression of certain immuno-modulatory proteins and numbers of particular cell types in CPPS patient samples, compared to controls. Studies have involved samples from various urogenital and systemic sources including EPS, seminal plasma, voided bladder 3 (VB3), urine, and semen. These studies have demonstrated increased levels of numerous pro-inflammatory cytokines including, IL1b, TNF-alpha, IL6, IL8 and IgA, while additional studies have described decreased levels of IL2R (T-cell development) in CPPS patients compared to controls. In terms of cellular infiltrate, it has been shown using multiple samples (EPS/VB3/semen) that there are increased numbers of granulocytes, macrophages and both activated T and B lymphocytes in CPPS patients. Prostate inflammation has also been examined histologically and it has been demonstrated that there is increased leukocyte infiltration and increased numbers of T-cells and more specifically CD8+ T cells compared to controls. The T-cell immune response to prostate antigens has also been investigated and it has been shown that CD4 T cells responded to seminal plasma from patients in two independent studies. Moreover IFN-gamma producing Th1 cells that have been identified in CPPS patient peripheral blood that are specific to prostate specific antigen (PSA). High levels of PSA in blood is commonly associated with prostate tissue damage and carcinogenesis. Another similar study identified IgA antibodies specific to two additional prostate antigens, MAD-PRO-34 and Ny-Co-7, in CPPS patients. These studies and others point to auto-reactivity to prostate antigen as a driver of CPPS and point to the existence of organ resident auto-reactive T-cells, that become activated following an initiation event.

Experimental autoimmune prostatitis (EAP) has consistently been used to determine the underlying immune responses associated with development of pain in mice. This model that has been used for the past 20 years to delineate possible mechanisms underlying pathogenesis in humans. Using such models, experiments have demonstrated that a particular antigen, prostatic steroid binding protein (PSBP)/prostatein is capable of inducing EAP, when injected with adjuvant alone, and it was also shown that additional antigens may be present in rat prostate homogenates to augment these effects. The model used in the experiment conducted during development of embodiments herein used a 1:1 ratio of rat prostate homogenate with adjuvant that is injected subcutaneously and pain is assessed by tactile allodynia after seven days, for up to 30 days. This model has been examined in multiple mouse strains, revealing that the development of pain occurs at the highest level, without the need for a second injection of antigen: adjuvant in the NOD background. Tactile allodynia of the pelvic region, a surrogate for pelvic pain has been used to study pelvic pain conditions in mouse models of CP/CPPS and closely related disease conditions such as interstitial cystitis.

Mast cells are a major focus of current research as the main mediator and effector cell in allowing disease to progress from initiation stages to breaking of tolerance, neuronal activation and eventually neuronal sensitization. Mast cells are derived from CD34 positive hematopoietic precursor cells in bone marrow. They circulate as immature cells, fully maturing only once resident at a specific tissue or site, it is then that they gain the potential to become activated. They are thought to be the first line of defense against infections by microbes and generally are closely associated with blood and lymphatic vessels and nerves. It has been shown that they demonstrate both protective and pathologic effects in a context dependent manner, e.g. wound healing, angiogenesis and inflammation. Crosslinking of their IgE receptor leads to hyper-sensitization as seen in conditions such as asthma. Upon activation, mast cells release several molecules stored in granules including, histamine, serotonin, tryptase, chymase, prostaglandin D2 (PGD2) and leukotriene B4 that set in motion early stage allergic responses. Independent of IgE crosslinking, activation of mast cells is classified as non-allergic and can again originate from a diverse range of sources including Pathogen Associated Molecular Patterns (PAMPs), complement, cytokines, drugs, hormones, and physical stressors such as temperature and pressure.

In some embodiments, provided herein are biomarkers (e.g., mast cell tryptase, carboxypeptidase A3 (CPA3), etc.), the expression, activity, and/or level of which correlates with CP/CPPS in a subject (e.g., level of one or more biomarkers in a sample (e.g., EPS, urine, etc.) above a threshold level indicates that the subject suffers from CP/CPPS). In some embodiments, a threshold, for example for mast cell tryptase in EPS, is 10 ng/ml, 15 ng/ml, 20 ng/ml, 25 ng/ml, 30 ng/ml, 35 ng/ml, 40 ng/ml, or values or ranges therebetween. In some embodiments, a level of a biomarker (e.g., mast cell tryptase, CPA3, etc.) in a sample (e.g., an EPS sample, a urine sample, etc.) from a subject above a threshold (e.g., 10 ng/ml, 15 ng/ml, 20 ng/ml, 25 ng/ml, 30 ng/ml, 35 ng/ml, 40 ng/ml, or values or ranges therebetween) indicates (e.g., is diagnostic of) that the subject suffers from CP/CPPS. Such markers find use in the diagnosis, detection, and/or characterization of CP/CPPS. In some embodiments, methods, assays, kits, reagents, and systems are provided for the detection and/or quantification of mast cell tryptase and CPA3 in a sample from a subject.

Experiments conducted during development of embodiments of the present invention demonstrate that mast cell tryptase is present, and significantly elevated, in EPS samples of men with CPPS. Thus, in some embodiments, the present invention provides reagents for quantification of mast cell tryptase in EPS for the diagnosis and treatment of CPPS. In some embodiments, detecting the levels of mast cell tryptase in a sample (e.g., EPS) permits diagnosis of or increased likelihood of CP/CPPS. In some embodiments, mast cell tryptase is used in order to further understand and characterize the etiology and pathogenesis of CPPS. In some embodiments, presence of or levels of mast cell tryptase is detected in a sample (e.g., EPS). In some embodiments, EPS is obtained from a subject (e.g., a patient) by accepted methods in the field. In some embodiments, the present invention provides detection of and/or measurement of mast cell tryptase as indicative of CP/CPPS. In some embodiments, the present invention provides diagnosis, characterization (e.g. identification of subtype), identification, etc. of CP/CPPS based on detection of mast cell tryptase. In some embodiments, the present invention provides diagnosis, characterization (e.g. identification of subtype), identification, etc. of CP/CPPS based on detection of mast cell tryptase above a threshold level (e.g. above a defined concentration in EPS). In some embodiments, patients are categorized (e.g., as IIIa and IIIb) and therapies or other interventions selected according to the levels of mast cell tryptase detected.

Experiments conducted during development of embodiments of the present invention demonstrate that carboxypeptidase A3 (CPA3) is present, and significantly elevated, in urine samples of men with CPPS. Thus, in some embodiments, the present invention provides reagents for quantification of CPA3 in urine for the diagnosis and treatment of CPPS. In some embodiments, detecting the levels of CPA3 in a sample (e.g., urine) permits diagnosis of or increased likelihood of CP/CPPS. In some embodiments, CPA3 is used in order to further understand and characterize the etiology and pathogenesis of CPPS. In some embodiments, presence of or levels of CPA3 is detected in a sample (e.g., urine). In some embodiments, urine is obtained from a subject (e.g., a patient) by accepted methods in the field. In some embodiments, the present invention provides detection of and/or measurement of CPA3 as indicative of CP/CPPS. In some embodiments, the present invention provides diagnosis, characterization (e.g. identification of subtype), identification, etc. of CP/CPPS based on detection of CPA3. In some embodiments, the present invention provides diagnosis, characterization (e.g. identification of subtype), identification, etc. of CP/CPPS based on detection of mast cell tryptase above a threshold level (e.g. above a defined concentration in urine). In some embodiments, patients are categorized (e.g., as IIIa and IIIb) and therapies or other interventions selected according to the levels of CPA3 detected.

In some embodiments, mast cell tryptase and/or CPA3 are detected in conjunction with other biomarkers (e.g. chemokines (e.g., MIP-1α and MCP-1)). In some embodiments, mast cell tryptase and/or CPA3 are detected individually (e.g., without other biomarkers). In some embodiments, detection of mast cell tryptase and/or CPA3 enhances the sensitivity of predicting and classifying CP/CPPS) over other methods known to those in the art. In some embodiments, detection of mast cell tryptase and/or CPA3, is used in combination with detection of other markers (e.g. chemokines/cytokines), including, but not limited to MIP-1α, MCP-1,GMCSF, IL-1, IL-2, IL-3, IL-4, IL-5, IL-6, IL-7, IL-8, IL-9, IL-10, IL-11, IL-12, IL-13, IL-14, IL-15, α-inteferon, γ-interferon, GRO-α, RENTES, fractalkine, VEGF, and TNF, in order to detect or classifyCP/CPPS. In some embodiments, detection of mast cell tryptase, is used in combination with symptoms and/or other known diagnostic/classification criteria to detect or classify CP/CPPS.

In some embodiments, the present invention provides methods for detection of expression biomarkers. In preferred embodiments, expression is measured directly (e.g., at the RNA or protein level). In some embodiments, expression and/or biomarker level is detected in samples (e.g., semen, seminal fluid, seminal plasma or expressed prostatic secretions). In other embodiments, expression and/or biomarker level is detected in bodily fluids (e.g., including but not limited to, plasma, serum, whole blood, mucus, and urine). The present invention further provides panels and kits for the detection of markers. In preferred embodiments, the presence of a CPPS marker is used to provide a prognosis to a subject. For example, the detection of mast cell tryptase and/or CPA3 in samples is indicative of CPPS. The information provided is also used to direct the course of treatment. For example, if a subject is found to have a biomarker level indicative of CP/CPPS, therapies can be started immediately in place of or in addition to other treatments that would have otherwise been used. In addition, if a subject is found to have a CPPS that is not responsive to other therapies, the expense and inconvenience of such therapies can be avoided.

The present invention is not limited mast cell tryptase and/or CPA3 as a marker. Any suitable marker that correlates with CPPS may be utilized, including but not limited to, MIP-1a, MCP-1, GMCSF, IL-1, IL-2, IL-3, IL-4, IL-5, IL-6, IL-7, IL-8, IL-9, IL-10, IL-11, IL-12, IL-13, IL-14, IL-15,α-inteferon, γ-interferon, GRO-α, RENTES, fractalkine, VEGF, and TNF. Additional markers are also contemplated to be within the scope of the present invention. Any suitable method may be utilized to identify and characterize markers suitable for use in the methods of the present invention. For example, in some embodiments, markers identified as being up or down-regulated in CPPS, e.g., using gene expression microarray methods, are further characterized using tissue microarray, immunohistochemistry, Northern blot analysis, siRNA or antisense RNA inhibition, mutation analysis, investigation of expression with clinical outcome, etc.

In some embodiments, CPPS biomarkers are detected by measuring the expression of corresponding mRNA in a sample. mRNA expression may be measured by any suitable method (hybridization, amplification, mass, sequencing, etc.). In other embodiments, gene expression of CPPS markers is detected by measuring the level of a protein or polypeptide. Protein level may be detected by any suitable method. In some embodiments, proteins are detected by immunohistochemistry or ELISA. In some embodiments, proteins are detected by their binding to an antibody raised against the protein. In some embodiments, the amount of a protein or other biomarker in a sample is detected by assaying for the activity of the protein or biomarker.

In certain embodiments, the present invention provides kits for the detection and characterization of CP/CPPS. In some embodiments, the kits contain antibodies specific for a CPPS marker (e.g., mast cell tryptase, CPA3, etc.), in addition to detection reagents and buffers. In other embodiments, the kits contain reagents specific for the detection of mRNA or cDNA (e.g., oligonucleotide probes or primers). In preferred embodiments, the kits contain all of the components necessary, sufficient, or useful to perform a detection assay, including all controls, directions for performing assays, and any necessary software for analysis and presentation of results.

In some embodiments, methods of treating CP/CPPS are provided herein (e.g., following testing for biomarkers and/or diagnosis of CP/CPPS with the methods herein). Suitable treatments for CP/CPPS within the scope herein include antibiotics, anti-inflammatory medications, muscle-relaxing medicines, alpha blockers, sitz bath, prostate massage, dietary changes, biofeedback, surgery, physical therapy, herbal treatment, mast cell stabilizer (e.g., cromolyn sodium), and histamine receptor antagonist (e.g., cetirizine).

In some embodiments, a subject is treated with a combination therapy comprising a mast cell stabilizer and a histamine receptor antagonist (See, e.g., U.S. Pat. Pub. No. 2022/004,0143; incorporated by reference in its entirety). For example, the mast cell stabilizer may be cromolyn sodium. In some embodiments, the histamine receptor antagonist is a histamine receptor 1 antagonist or a histamine receptor 2 antagonist. In some embodiments, the histamine receptor 1 antagonist is cetirizine.

In some embodiments, provided herein are compositions and methods for detection, diagnosis, treatment and/or prevention of chronic pelvic pain syndrome, biomarkers of chronic pelvic pain syndrome (e.g., mast cell markers (e.g., tryptase)), and/or inhibition of mast cell function (e.g. inhibition of MCP-1 and/or MIP-1a) to treat or prevent chronic pelvic pain syndrome, such as those understood in the field (See, e.g., U.S. Pat. No. 8,586,044; incorporated by reference in its entirety).

### EXPERIMENTAL

### Example 1

EAP is Associated with Increased Mast Cell Density and Mast Cell Activation

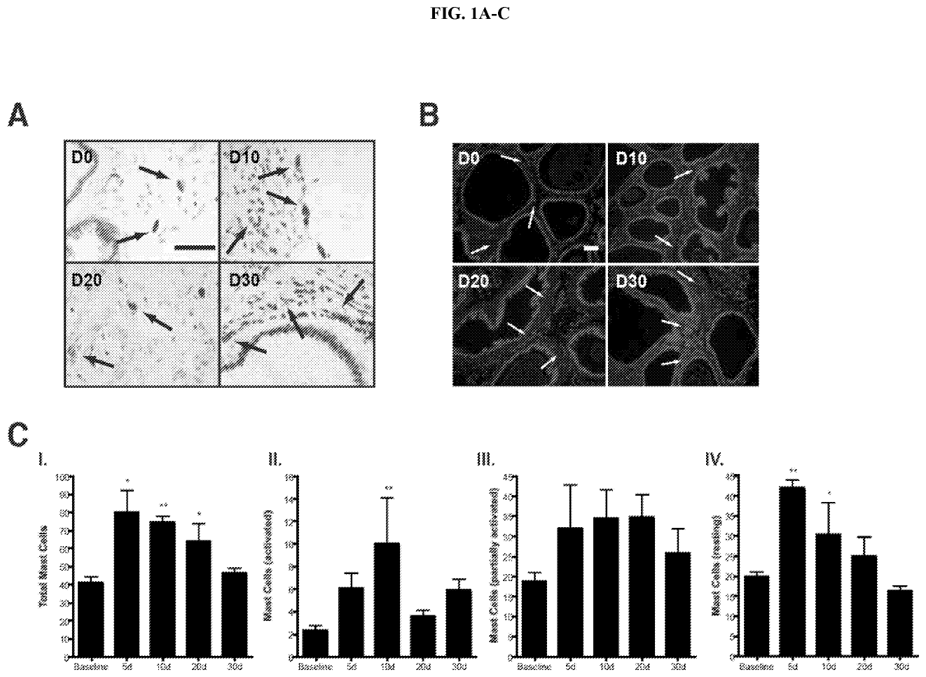

Experiments were conducted during development of embodiments herein to study mast cells in a murine autoimmune prostatitis model of chronic pelvic pain. Mast cells were identified by toluidine blue staining and individually counted as total, resting, partially activated or fully activated depending on the dispersal of toluidine blue stained granules (FIG. 1A). Total mast cells were increased 5 days after induction of EAP with the majority of cells in the resting stage (FIG. 1C). By day 10 there was significant activation of mast cells that was not observed at 20 and 30 days. However, the apparent reduction in resting cells at 20 and 30 days combined with a lack of any increase in activated cells indicates that a portion of late stage activated cells are not detected because of degranulation. In addition, prostate sections were examined from 0 (baseline), 10, 20 and 30 days after the initiation of autoimmunity for the expression of NGF, a product of mast cell degranulation (FIG. 1B). NGF was observed to increase at days 10 and 20 with maximal expression being present at day 30 corresponding to the potential period of maximal mast cell degranulation.

### Example 2

Mast Cell Deficiency is Associated with an Attenuation of Pelvic Pain in EAP.

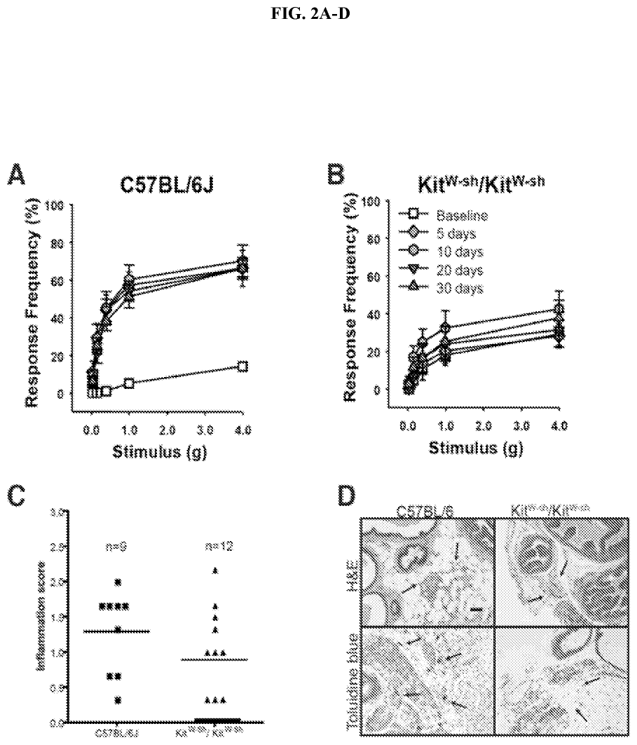

Experiments were conducted during development of embodiments herein to examine the role of mast cells in EAP pathogenesis by utilizing adult KitW-sh/KitW-sh mice (5-7 weeks of age) that were deficient in tissue mast cells due to a mutation in the c-kit receptor. Autoimmune prostatitis was induced in KitW-sh/KitW-sh and wild-type C57BL/6 (B6) mice followed by study of pelvic pain development as indicated by enhanced tactile allodynia of the suprapubic region. B6 mice developed robust pelvic pain five days after antigen administration that persisted up to thirty days (FIG. 2A). In contrast, KitW-sh/KitW-sh mice did not show any increase in pelvic pain behavior at 5 days and showed inhibited pain responses at 10, 20 and 30 days after antigen administration (FIG. 2B). To assess the specificity of EAP-induced tactile sensitivity, the 50% threshold sensitivity was quantified in the paw. EAP induced no significant changes in tactile sensitivity of the plantar region of the hind paw in either strain of mice. There was also no prolonged change in weight suggesting that EAP in the B6 and KitW-sh/KitW-sh mice is not associated with changes in gross physiology.

Experiments were conducted during development of embodiments herein to quantify the extent of inflammation in the prostates of B6 and KitW-sh/KitW-sh mice following EAP induction. Inflammation in both groups of mice was chronic with multiple foci of inflammatory cells distributed predominantly in the periglandular regions and stroma of the prostate (FIG. 2D, top panels). Both groups of mice developed chronic inflammation that was not statistically different when quantified using standard inflammation scoring (FIG. 2C). In contrast, toluidine blue staining for mast cells revealed large numbers of mast cells in various stages of activation in the stroma of C57BL/6 but not KitW-sh/KitW-sh mice (FIG. 2D, lower panels). Thus, mast cells do not appear to be a requirement for the development of inflammation per se but appear to play a critical role in the establishment of chronic pain.

### Example 3

CP/CPPS is Associated with Elevated Levels of Mast Cell Mediators in EPS and Urine

Mast cells have been postulated to play an important role in the pathogenesis of CP/CPPS in clinical patients and in animal models. It has previously been demonstrated that mast cell tryptase, a product of mast cell degranulation, is elevated in patients with CP/CPPS. Experiments were conducted during development of embodiments herein to examine expressed prostatic secretions (EPS) and urine samples obtained immediately prior (VB2) and immediately after prostatic massage (VB3) from patients and healthy volunteers for the presence of the mast cell degranulation products, tryptase, carboxypeptidase A (CPA3) and NGF. VB2 urines were considered to be representative of mast cell products released from the bladder and urethra and VB3 as representative of products secreted from the prostate, in addition to those from the bladder and urethra of men. The EPS and VB2 subtracted VB3 (VB3-VB2, prostate specific release) levels of tryptase-β, CPA3 and NGF were compared between CP/CPPS and healthy volunteers. The presence of mast cell tryptase was measured using a colorimetric substrate-based assay as an index of mast cell degranulation

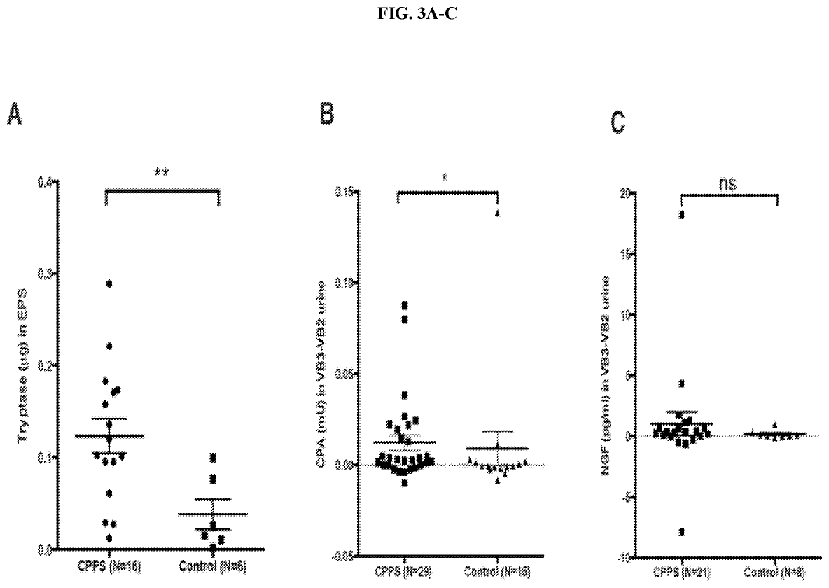

The calorimetric assay was based on the spectrophotometric determination of the chromophore p-nitroaniline following the action of tryptase on the substrate tosyl-gly-pro-lys-pNA. In all cases, EPS samples drawn from the different patient cohorts were thawed on ice and a small aliquot was withdrawn for tryptase determination. Relative tryptase activity was determined by simultaneous inclusion of a tryptase positive standard in the experiment. Significantly elevated levels of tryptase was observed in the EPS of CP/CPPS patients (FIG. 3A) compared to healthy controls (p=0.0060). CPA3, a mast cell carboxypeptidase that acts with tryptase-β and is released upon mast cell activation and degranulation was significantly elevated in CP/CPPS (FIG. 3B) urines (VB3-VB2) compared to healthy volunteers (p=0.0359). NGF, a secreted protein from mast cells that is known to be elevated in similar pain conditions such as interstitial cystitis, was also examined. No significant difference between CP/CPPS and control urines was detected for NGF levels (FIG. 3C). These data demonstrate that degranulation products of mast cells, CPA3 and tryptase-β are associated with CP/CPPS.

### Example 4

CP/CPPS Patients with Elevated Mast Cell Tryptase Levels Showed Significant Symptom Relief with Combination Mast Cell Inhibitor

### Study Population

The clinical study population consisted of males diagnosed with Category III CP/CPPS reporting pain and discomfort in the pelvic region for at least a 3-month period within the 6 months immediately before the first baseline screening visit. Subjects were screened for increased active mast cell tryptase levels in their prostatic fluid. Men who met the inclusion and exclusion criteria were enrolled into the study. Subjects were administered a three-week course of cromolyn sodium and cetirizine dihydrochloride.

### Data and Specimen Collection

Chronic Prostatitis Symptom Index (NIH-CPSI) questionnaire data, Voided Bladder 3 (VB3), and expressed prostatic secretions (EPS) samples were collected. The questionnaire was designed specifically to assess patient scored symptoms of CPPS and divides score into three areas: Pain, Urinary and Quality of life as described under NIH-CPSI symptoms score. VB3 samples and EPS samples were either used immediately or stored at −80° ° C. for future analyses.

### NIH-CPSI Symptom Score and AUA-Symptom Index

The NIH-CPSI and AUA-BPH index was collected for each participant in the study. The NIH-CPSI contains 13 items focusing on the 3 domains of pain or discomfort (8 items), urinary symptoms (2 items) and quality of life impact (3 items). By summing the pain items a sub-score of 0 to 21 was derived for the study participants. Similarly, a urinary sub-score of 0 to 10 was derived and a quality-of-life impact sub-score of 0 to 12 was created. In addition, an overall NIH-CPSI score of 0 to 43 was calculated per participant by summing the pain (range 0 to 21), urinary (range 0 to 10) and quality-of-life impact (range 0 to 12) sub-scores.

Mast Cell Tryptase Quantitation Mast cell tryptase levels was measured in EPS from CPPS patients using the mast cell degranulation kit (Millipore) following manufacturer's protocol.

### Results

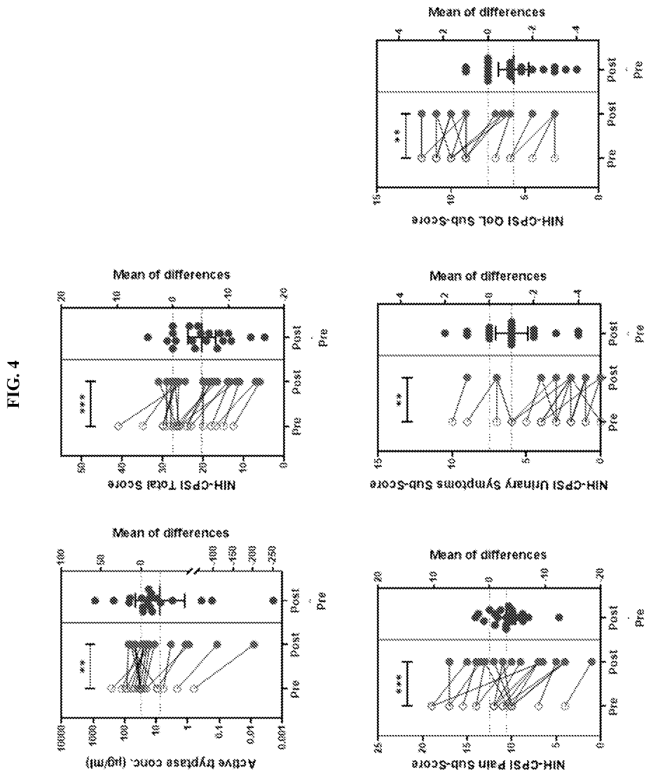

Analysis of expressed prostatic secretions from 19 out of 20 patients showed a significant reduction in active tryptase at the end of the trial (FIG. 4). In evaluating questionnaire data from the NIH-CPSI (20 out of 20), a statistically significant reduction in the total NIH-CPSI score as well as separate significant reductions in the pain, urinary and quality of life sub-scores was observed. These results indicate that CP/CPPS patients with elevated mast cell tryptase may derive substantial clinical benefit from combination mast cell inhibitor therapy. Furthermore, the reduction in symptoms following treatment was associated with a reduction in mast cell tryptase levels in EPS.

1. A method comprising:
(a) obtaining an expressed prostatic secretion (EPS) sample from a subject;
(b) having the sample tested to quantify the level of mast cell tryptase in the sample.

2. The method of claim 1, further comprising diagnosing the subject as suffering from chronic prostatitis (CP) and/or chronic pelvic pain syndrome (CPPS) if the tested level of mast cell tryptase in the sample is elevated above control.

3. The method of claim 1, further comprising treating the subject for CP/CPPS if the tested level of mast cell tryptase in the sample is elevated above control.

4. The method of claim 3, wherein the treatment comprises a mast cell inhibitor therapy.

5. The method of claim 4, wherein the mast cell inhibitor therapy comprises administration of cromolyn sodium and/or cetirizine dihydrochloride

6. A kit comprising reagents for performing the method of claim 1.

7. A method comprising:
(a) obtaining a urine sample from a subject;
(b) having the sample tested to quantify the level of carboxypeptidase A3 (CPA3) in the sample.

8. The method of claim 7, further comprising diagnosing the subject as suffering from chronic prostatitis (CP) and/or chronic pelvic pain syndrome (CPPS) if the tested level of carboxypeptidase A3 (CPA3) in the sample is elevated above control.

9. The method of claim 7, further comprising treating the subject for CP/CPPS if the tested level of carboxypeptidase A3 (CPA3) in the sample is elevated above control.

10. A kit comprising reagents for performing the method of claim 7.

11. A method comprising the steps of:
(a) having a urine sample from a subject tested to quantify the level of carboxypeptidase A3 (CPA3) in the sample; and
(b) having an EPS sample from a subject tested to quantify the level of mast cell tryptase in the sample.

12. The method of claim 11, further comprising diagnosing the subject as suffering from chronic prostatitis (CP) and/or chronic pelvic pain syndrome (CPPS) if the tested level of mast cell tryptase and/or the level of CPA3 is elevated above control.

13. The method of claim 11, further comprising treating the subject for CP/CPPS if the tested level of mast cell tryptase and/or the level of CPA3 is elevated above control.

14. A kit comprising reagents for performing the method of claim 7.

15. A method comprising:
(a) obtaining a biological sample from a subject;
(b) having the sample tested to quantify the level of mast cell tryptase in the sample.

16. The method of claim 15, wherein the biological sample is an expressed prostatic secretion (EPS) sample.

17. The method of claim 15, wherein the subject is suspected of suffering from chronic prostatitis/chronic pelvic pain syndrome (CP/CPPS).

18. The method of claim 15, further comprising diagnosing the subject as suffering from chronic prostatitis (CP) and/or chronic pelvic pain syndrome (CPPS) if the tested level of mast cell tryptase in the sample is elevated above control.

19. The method of claim 15, further comprising treating the subject for CP/CPPS if the tested level of mast cell tryptase in the sample is elevated above control.

20. A kit comprising reagents for performing the method of claim 15.
